(19) United States
(12) Patent Application Publication
(10) Pub. No.: US 2025/0127803 A1
(43) Pub. Date: Apr. 24, 2025

(54) 2'-FUCOSYLLACTOSE AND LACTO-N-NEOTETRAOSE FOR PREVENTING AND TREATING OVERWEIGHT

(71) Applicant: HEALTH AND HAPPINESS (H&H) HONG KONG LTD, Hong Kong (CN)

(72) Inventor: Lane Jonathan, Cork (IE)

(21) Appl. No.: 18/835,677

(22) PCT Filed: Feb. 4, 2022
(86) PCT No.: PCT/IB2022/050979

§ 371 Date: Aug. 2, 2024

(51) Int. Cl. A61K 31/702; A61P 3/04

(52) U.S. Cl. CPC A61K 31/702; A61P 3/04

(57) ABSTRACT

It relates to the use of 2′-fucosyllactose and Lacto-N-neotetraose for treating and/or reducing weight in a subject in need thereof. It also relates to pharmaceutical and food compositions containing these compounds.

### TECHNICAL FIELD

The present invention relates to the field of weight reduction. In particular the present invention relates to the use of two compounds, 2′-fucosyllactose and lacto-N-neotetraose, for reducing weight in a subject in need thereof.

### BACKGROUND

Overweight is an overall aspect in societies of industrialized countries which is generally not considered to meet the beauty standard of the 21^{st} century. In 2019 about 65% of the adult population in the United States was considered overweight, while this rating applied to about 53% of the population of the European Union. In China meanwhile about 50% of the adult population is deemed to exceed the normal weight. Common to all of these countries or areas, respectively, is that in the recent year more and more children become overweight, with risk of obese condition(s).

Obesity is an associated risk factor with a number of different diseases which can result in higher morbidity and, eventually, higher mortality. Diseases often observed in obese individuals are diabetes mellitus (type 2), insulin resistance syndrome, cardiovascular diseases, hypertension, atherosclerosis, congestive heart failure, stroke, gallbladder disease, osteoarthritis, sleep apnoea, reproductive disorders such as polycystic ovarian syndrome. Also, a higher incidence of cancer such as breast cancer and prostate cancer has been postulated to be related with obesity.
Especially in view of these health issues there is a general desire to reduce body weight. Attempts to achieve this goal by the individual itself comprise resorting to a diet which does not provide sufficient calories to the body, radically changing the nature of food ingested, e.g. omitting any meat or having a meal only after extended periods of time, e.g. after 16 hours only. All of these measures are cumbersome and disliked by the individuals, which have to change their life-style.

Also, the food industry found this problem as a promising new market proposing a number of different ingredients assisting individuals to maintain or even decrease weight. Among these ingredients are agents inhibiting the deposit of fat in the cells or by increasing lipid oxidation rates. Another focus was directed to agents diminishing appetite.

Also, the pharmaceutical industry focused on this problem providing drugs that prevent calorie uptake from the food, a famous representative thereof being Orlistat®, an agent blocking lipases in the gut. Yet, there are side effects with Orlistat® leading in some cases to fluidized feces. Also, as a synthetic molecule, Orlistat®, has other side effects since not naturally designed to be in the body.

There remains a need to provide new agents, which are safe to consume or administer and which are effective in reducing weight of an overweight or obese person.

### SUMMARY OF THE INVENTION

In order to solve the above problem, the present invention provides 2′-fucosyllactose and/or lacto-N-neotetraose for use in the treatment or prevention of overweight, obesity and/or any related condition associated therewith.

2′-fucosyllactose and lacto-N-neotetraose are both members of a family of glycans found in human milk and termed Human Milk Oligosaccharides (HMOs). 2′-fucosyllactose is the most prevalent HMO and may account for up to 30% of all HMOs in the breast milk. In general, 2′-fucosyllactose has a protective effect against infectious diseases, by preventing adherence of toxins or pathogens to the gut epithelium and is associated with promoting infant growth. On the other hand, lacto-N-tetraose as another member of HMOs is a complex sugar enzymatically synthesized from the substrate lactose. It is biologically relevant in the early development of the infant gut flora and presently considered as a prebiotic, promoting the growth of healthy bacteria, such as Bifidobacterium longum subspecies infantis, in the gut microbiome.

In the course of extensive studies leading to the present invention the inventors have investigated the ability of nematode worm C. elegans to accumulate lipids under exposure to different agents. Despite its apparent simplicity, C. elegans is an acknowledged model for research, particularly in the functional characterization of novel gene functions and drug targets that have been identified using proteomic and genomic technologies. The cellular complexity and shared heritage of disease pathways of C. elegans and higher organisms, together with the simplicity and cost-effectiveness of cultivation, has admittedly placed this organism at center stage as an effective in vivo model that is amenable to whole-organism screens and target validation.

It has now surprisingly been found that specific concentrations of 2′-fucosyllactose or lacto-N-neotetraose are effective in reducing the lipid level in this organism. In fact it has been found that 2′-fucosyllactose is about 70-90% as effective as is (25)-2-Formylamino-4-methylpentansäure-(1S)-1-{[(35,4S)-3-hexyl-2-oxo-4-oxetanyl]methyl}dodecylester, a compound sold under the tradename Orlistat® and acknowledged to be a potent drug for the treatment of obesity. Also, lacto-N-neotetraose has been found to be about 40-60% as effective for reducing the lipid content as is Orlistat® taken as a reference.

The invention also relates to a composition containing an effective amount 2′-fucosyllactose, lacto-N-neotetraose or a combination thereof for use in treating and/or preventing overweight, obesity and/or any related conditions in a subject in need thereof.

### FIGURES

FIG. 1 shows the fat reducing effect of 2′-fucosyllactose in C. elegans at various concentrations.

FIG. 2 shows the fat reducing effect of lacto-N-neotetraose in C. elegans at various concentrations.

### DETAILED DESCRIPTION OF THE INVENTION

The invention relates to 2′-fucosyllactose and/or lacto-N-neotetraose for use in treating or preventing overweight, obesity and/or related diseases in a subject. 2′-fucosyllactose has first been discovered in human milk and isolated therefrom in the 1950s and methods for its isolation are well known in the art. Since human milk is inaccessible in large amounts and its separation from the complex mixture milk is cumbersome, the compound may also by chemically synthesized or even produced in recombinant E. coli by known means.

The subject to which the compounds are to be administered are mammals, in particular humans or pets.

The invention further provides a composition comprising an effective amount of 2′-fucosyllactose and/or lacto-N-neotetraose for use in treating overweight, obesity and/or any related diseases in a subject. In the context of the invention the phrase an effective amount shall designate an amount which when administered to a subject brings about the desired effect, i.e. interferes with the lipid metabolism such that a reduction of weight in the individual may be observed. In principle a daily dosage for humans an amount of from about 10 mg/kg body weight to about 90 mg/kg body weight are envisaged, preferably from about 15 mg/kg body weight to about 65 mg/kg body weight of 2′-fucosyllactose more preferably from about 25 mg/kg body weight to about 40 mg/kg body weight. For lacto-N-neotetraose an daily dosage of from about 5 mg/kg to about 50 mg/kg are envisaged, preferably from about 8 mg/kg body weight to about 40 mg/kg body weight, more preferably from about 12 mg/kg body weight to about 20 mg/kg body weight.

The composition proposed in the present invention may contain any of the respective compounds individually or a combination of both, 2′-fucosyllactose and/or lacto-N-neotetraose.

The composition proposed in the present invention may be provided as a pharmaceutical composition or as a food composition.

When provided as pharmaceutical composition, the composition may further comprise conventional agents designed for producing or shaping the product, providing a targeted release or for protecting the compounds from the environmental conditions exposed when moving through the gastrointestinal tract until resorption. Non-limiting examples for these agents are filling agents, solvents, emulsifier, buffer, thickeners, coatings, disintegrants, lubricating agents, flowing agents, preservatives, resorption accelerating agents etc.

The pharmaceutical composition may be suitable for oral administration and may be in a form selected from the group comprising tablets, dragees, capsules, granules, powders, syrups, aqueous suspensions or aqueous solutions. Alternatively, the composition may be in form of a depository.

The pharmaceutical composition may be used together with other agents known to reduce and/or prevent overweight and/or obesity and related diseases, such as Orlistat®, 5-Hydroxytryptophan, Lorcaserin, and Bupropion/Naltrexone. The additional agents may be provided separately or may be included in the composition illustrated herein.

In the context of the present invention the phrase related diseases comprises any of diseases or conditions, for which overweight or obesity is a known risk factor. Non-limiting examples are metabolic syndrome, glycaemia, diabetes type 2, hypercholesterolemia, cardiovascular diseases, hypertension, inflammation, hormonal alterations, infertility, etc.

The composition proposed here may also be in form of a food composition. In the context of the present invention the term food composition comprises anything of a meal itself, an additive or supplement etc., Due to the nature of the compounds 2′-fucosyllactose and/or lacto-N-neotetraose, both natural milk oligosaccharides, the compounds or compounds, respectively, may be admixed to any food material ingested by an individual without substantive side effects to be expected. As such the compounds may be added to any conventional food product, such as juices of fruit or vegetable source, milk products, such as milk, jogurt, kefir, cheese, ice cream, soft drinks, pastries etc. The compounds may also be formulated as nutritional supplements containing the oligosaccharides in concentrated form and as such in higher doses allowing a higher absorption thereof. Likewise, the food composition may also be designed for animals, for example pets, wherein the compounds as illustrated herein are mixed into conventional pet food in any amount with the proviso of not deterioration the physical appearance and taste of the pet food.

According to an embodiment the food composition as illustrated above may also be used together or complemented by other known weight loss supplements or agents, respectively, such as Stivia® or microcellulose.

Also, the present invention provides the use of 2′-fucosyllactose and/or lacto-N-neotetraose and/or a composition containing these compounds for reducing weight of a non-obese person.

### EXAMPLES

### Example 1: Fat-reducing Effect of 2′-Fucosyllactose on C. elegans

Plates with a diameter of 100×15 mm have been filled with NGM (Nematode Growth Medium, as standard, hereafter NG medium (Fisher Scientific). Nile red (Sigma, St. Louis, MO, USA) was added to the solid surface of the NGM plates seeded at a final concentration of 0.05 μg/mL

For each of the experiments 2 NG medium plates were used, which had been supplemented with 2′-fucosyllactose in different amounts each, i.e. 0.01, 0.1, 1, 10 and 30 mg/mL. As negative control two plates with NG medium were used. As positive control two NG medium plates with Orlistat® were used.

60 synchronized worms at young adult stage were added to each plate with Escherichia coli strain OP50 as a food source and cultured under aerobic conditions at 20° C.

All worms were subjected to a lipid staining method, following the protocol previously described (Martorell et al., 2016).

All nematodes from each plate were transferred to M9 buffer and the fluorescence was measured using a FP-8300 system (JASCO Analytical Instrument, Easton, MD, USA). Fat deposition in worms was determined by one-way ANOVA test, using a Tukey's multiple comparison post-test.

Results are given as the mean±standard deviation. The statistical analyses were performed with GraphPad Prism 9 software. Statistical significance is indicated at 5%.

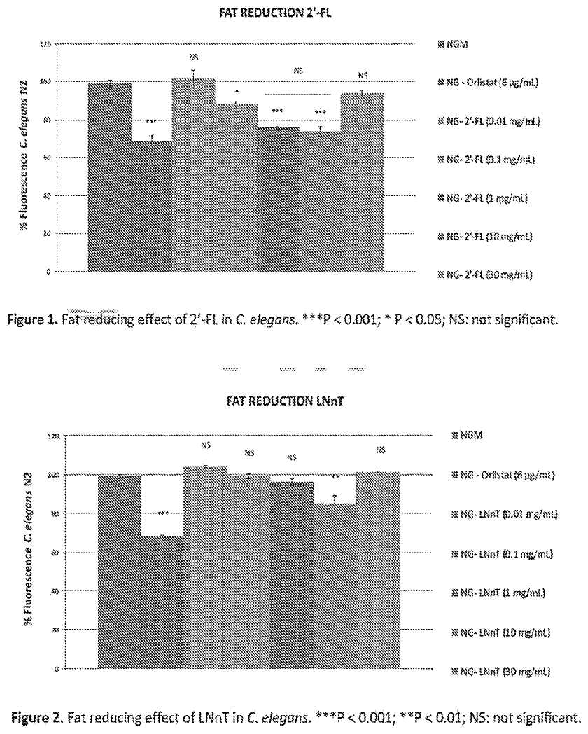

The results are shown in Table 1 and FIG. 1.

TABLE 1
Percentage of fat reduction of 2′-fucosyllactose (2′-FL)
at different doses versus control feeding (NG medium)

| | Fat Reduction (%) vs. | |
|---|---|---|
| Condition | Control | Statistical |
| Orlistat | 30.40 | P < 0.001 |
| (6 μg/mL) | | |
| 2′-FL | −2.38 | Not |
| (0.01 mg/L) | | significant |
| 2′-FL | 11.37 | P < 0.05 |
| (0.1 mg/L) | | |
| 2′-FL (1 mg/L) | 23.61 | P < 0.001 |
| 2′-FL (10 mg/L) | 25.35 | P < 0.001 |
| 2′-FL (30 mg/L) | 5.28 | Not |
| | | significant |

Results show dose-dependency of fat reduction to the applied 2′-FL. The lower and higher doses resulted in no functional effect, while the medium doses (0.1, 1 and 10 mg/mL, see FIG. 1, purple and dark green column) significantly reduced fat content in nematodes, providing 23.6 and 25.3% of fat reduction compared to negative control conditions (NG medium). This effect was similar to that of the positive control orlistat (see FIG. 1, red column), which resulted in 30.4% fat reduction compared to negative control. Thus, the most effective doses 1 and 10 mg/ml of 2′-FL provided about 78% and 83% of the effectiveness of the positive control orlistat.

### Example 2: Fat-Reducing Effect of Lacto-N-Neotetraose on C. elegans

The Assay of example 1 has been repeated with the proviso that lacto-N-neotetraose has been used (doses 0.01, 0.1, 1, 10 and 30 mg/mL) instead of 2-fucosyllactose.

The results are shown in Table 2 and FIG. 2.

TABLE 2
Percentage of fat reduction of Lacto-N-neotetraose (LNnT)
at different doses versus control feeding (NG medium)

| | Fat Reduction (%) vs. | |
|---|---|---|
| Condition | Control | Statistical |
| Orlistate | 31.14 | P < 0.001 |
| (6 μg/mL) | | |
| LNnT | −5.01 | Not |
| (0.01 mg/L) | | significant |
| LNnT | −0.08 | Not |
| (0.1 mg/L) | | significant |
| LNnT (1 mg/L) | 3.08 | Not |
| | | significant |
| LNnT | 14.05 | P < 0.01 |
| (10 mg/L) | | |
| LNnT | −2.39 | Not |
| (30 mg/L) | | significant |

The results show that worms cultured in conditions containing 10 mg/mL of lacto-N-neotetraose (see FIG. 2, dark green column) had a fat content 14.05% lower than worms under negative control conditions (NGM). Orlistat, again serving as positive control in this approach, provided an effect of 31.14% fat reduction (see FIG. 2, red column). 10 mg/ml of lacto-N-neotetraose provided about 45% of the effect of the positive control.

1. 2′-Fucosyllactose and/or Lacto-N-neotetraose for use in preventing and/or treating overweight and/or obesity in a subject.

2. A composition comprising an effective amount of 2′-fucosyllactose and/or lacto-N-neotetraose for use in treating obesity and/or overweight in a subject.

3. The composition for use according to claim 2, comprising 2′-fucosyllactose and lacto-N-neotetraose.

4. The composition for use according to claim 2, wherein the composition is suitable for oral administration.

5. The composition for use according to claim 2, which is a pharmaceutical composition.

6. The composition for use according to claim 5, further comprising at least one carrier and/or a pharmaceutically acceptable excipient.

7. The composition for use according to claim 3 in the form of a tablet, a capsule, a powder, granules, a syrup, aqueous suspensions or solutions.

8. The composition for use according to claim 3 comprising an additional agent for reducing obesity and/or weight in a subject.

9. The composition for use according to claim 6, wherein the additional agent is Orlistat®, 5-Hydroxytryptophan, Lorcaserin, and Bupropion/Naltrexone.

10. The composition for use according to claim 2, which is a food composition.

11. The composition for use according to claim 10, selected from the group comprising fruit or vegetable juices, ice-cream, infant formula, milk, yogurt, cheese, fermented milk, powder milk, cereals, bakery products, milk and/or cereal-based products, nutritional supplements, soft drinks, dietary supplements or pet food.

12. The composition for use according to claim 10, wherein the composition is in form of a food additive of supplement.
